(19) United States
(12) Patent Application Publication
(10) Pub. No.: US 2025/0046471 A1
(43) Pub. Date: Feb. 6, 2025

(54) SYSTEM THAT EFFICIENTLY CALCULATES AND SETS ALARM THRESHOLDS FOR PATIENT MONITORING DEVICES

(71) Applicant: Nihon Kohden Digital Health Solutions, Inc., Irvine, CA (US)

(72) Inventors: Elizabeth BUDI, Irvine, CA (US); Allison AUSTIN, Folsom, CA (US); Harsh DHARWAD, San Diego, CA (US); Abel LIN, San Diego, CA (US); Timothy RUCHTI, Irvine, CA (US); Brian TU, Rosemead, CA (US); Arthur WEBB, Escondido, CA (US)

(73) Assignee: Nihon Kohden Digital Health Solutions, Inc., Irvine, CA (US)

(21) Appl. No.: 18/363,158

(22) Filed: Aug. 1, 2023

(51) Int. Cl. G16H 50/70; G16H 15/00; G16H 40/67

(52) U.S. Cl. CPC G16H 50/70; G16H 15/00; G16H 40/67

(57) ABSTRACT

A system that analyzes alarms from patient monitoring devices and calculates modified alarm thresholds that reduce the number of alarms to a desired level. This capability addresses the common problem of alarm overload and fatigue, where alarms occur so frequently that clinicians cannot respond effectively. The system supports highly efficient calculation of changes in alarm frequency, by storing summaries of alarm data that record maximum and minimum values during an alarm. New thresholds may be selected manually or automatically and may be transmitted directly to the patient monitoring devices as updates to their alarm thresholds. The system may also classify alarms as high (above an upper threshold) or low (below a lower threshold) when devices do not provide this data. The system may also estimate the number of additional alarms that would occur if an upper threshold were reduced, or a lower threshold were increased.

### BACKGROUND OF THE INVENTION

### Field of the Invention

One or more embodiments of the invention are related to the field of health care information systems and medical devices. More particularly, but not by way of limitation, one or more embodiments of the invention enable a system that efficiently calculates and sets alarm thresholds for patient monitoring devices.

### Description of the Related Art

Patients in medical facilities are often monitored by various devices that generate alarms when the values measured go outside certain configurable thresholds. In many facilities, alarms occur very frequently and may overload the clinical staff. Although facility management recognizes the problem of alarm overload, to date they have no effective tools to analyze the sources of excessive alarms and to systematically investigate the effect of modifying device alarm thresholds on the resulting number of alarms.

For at least the limitations described above there is a need for a system that efficiently simulates potential threshold scenarios and that efficiently calculates and sets alarm thresholds for patient monitoring devices.

### BRIEF SUMMARY OF THE INVENTION

One or more embodiments of the invention may enable a system that efficiently calculates and sets alarm thresholds for patient monitoring devices. The system may collect data from devices and analyze the alarms that occur over a time period to evaluate the impact of changing alarm thresholds on the number of alarms.

One or more embodiments of the invention may include a data collection system and an alarm threshold analysis system. The data collection system may have a first processor that is coupled to a database and coupled to multiple patient monitoring devices. Each patient monitoring device may be configured to obtain a time series of patient data samples, receive one or more threshold values, and when the patient data samples are outside the threshold values, generate an alarm and transmit alarm data to the first processor that includes the patient data samples while the alarm is active. The first processor may be configured to generate an alarm summary record associated with the alarm data and store it in the database. The alarm summary record may include the alarm, the alarm start time, the alarm duration, the minimum value of the patient data samples of the alarm data, and the maximum value of the patient data samples of the alarm data. The alarm threshold analysis system may have a second processor coupled to the database that is configured to retrieve the alarm summary records over a time period from the database, and to calculate the expected change in the number of alarms over the time period as a function of one or more modified threshold values based on the alarm summary records.

In one or more embodiments, the second processor may determine a new threshold value of the one or more modified threshold values and transmit this new threshold value to one or more of the patient monitoring devices to replace one or more of the threshold values. The new threshold value be a modified threshold value that results in a target value of the expected change in the number of alarms over the time period.

In one or more embodiments the second processor may also be configured to obtain or calculate a classification of each alarm summary record as either a high alarm corresponding to patient data samples above an upper threshold, or a low alarm corresponding to patient data samples below a lower threshold.

In one or more embodiments calculating the expected change in the number of alarms over the time period for a modified upper threshold may include eliminating alarm summary records with a maximum value of patient data samples below the modified upper threshold. Calculating the expected change in the number of alarms for a modified lower threshold may include eliminating alarm summary records with a minimum value of patient data samples above the modified lower threshold.

In one or more embodiments, the alarm summary records may further include the first value of patient data samples of the vital sign or metric of the alarm data, and calculating a classification of each alarm summary record into high or low alarms may include applying a k-means clustering algorithm with two clusters to a dataset that includes the first value of the alarm summary records, calculating a separation value as the mean of the centroids of the two clusters, and classifying each alarm summary record as a high alarm when the first value is above the separation and as a low alarm when the first value is below the separation value.

In one or more embodiments the second processor may also be configured to calculate one or both of the upper threshold and the lower threshold. This calculation may include calculating a first frequency distribution of the first values of alarm summary records classified as high alarms, a first modal frequency as the frequency of a mode of all or a portion of the first frequency distribution, a second frequency distribution of the first values of alarm summary records classified as low alarms, and a second modal frequency as the frequency of a mode of all or a portion of the second frequency distribution. The upper threshold may be calculated as the smallest value above the separation value having a frequency in the first frequency distribution that is greater than a first fraction times the first modal frequency, and the lower threshold may be calculated as the largest value below the separation value having a frequency in the second frequency distribution that is greater than a second fraction times the second modal frequency.

In one or more embodiments the second processor may also be configured to calculate the expected increase in the number of alarms over the time period when the upper threshold is decreased to a smaller upper threshold, or the lower threshold is increased to a larger lower threshold (or both).

In one or more embodiments calculating the expected increase in the number of alarms over the time period may include obtaining or estimating a distribution of patient data samples. When the upper threshold is decreased to smaller upper threshold, the expected increase in the number of alarms may be the frequency of the distribution between the smaller upper threshold and the upper threshold, divided by the frequency of the distribution above the upper threshold, multiplied by the number of alarms. When the lower threshold is increased to a larger lower threshold, the excepted increase in the number of alarms may be the frequency of the distribution between the lower threshold and the larger lower threshold, divided by the frequency of the distribution below the lower threshold, multiplied by the number of alarms.

In one or more embodiments, calculating the expected increase in the number of alarms over the time period may include calculating one or both of a first distribution of expected alarm maximum values based on extrapolation of a frequency distribution of the maximum values of the alarm summary records, and a second distribution of expected alarm minimum values based on extrapolation of a frequency distribution of the minimum values of the alarm summary records. When the upper threshold is decreased to a smaller upper threshold, the expected increase in the number of alarms may be calculated as the total frequency of the first distribution between the smaller upper threshold and the upper threshold. When the lower threshold is increased to a larger lower threshold, the expected increase in the number of alarms may be calculated as the total frequency of the second distribution between the lower threshold and the larger lower threshold.

In one or more embodiments, extrapolation of the frequency distribution of the maximum value and extrapolation of the frequency distribution of the minimum value may include linear regression.

In one or more embodiments, the first processor may also be configured to not store an alarm summary record when the alarm duration is below a duration threshold value.

### BRIEF DESCRIPTION OF THE DRAWINGS

The above and other aspects, features and advantages of the invention will be more apparent from the following more particular description thereof, presented in conjunction with the following drawings wherein:

FIG. 1 illustrates a problem addressed by one or more embodiments of the invention: a medical facility has many devices monitoring many patients, and the devices generate alarms when values are outside a defined range; unless alarm thresholds are set intelligently, alarms occur too frequently, and clinicians are unable to prioritize and respond effectively.

FIG. 2A shows an architectural diagram of an embodiment of the invention that addresses the problem of FIG. 1 by recording alarms and analyzing them to find more effective alarm thresholds.

FIG. 2B illustrates how the components of FIG. 2A may be integrated in one or more embodiments into an overall system architecture of healthcare devices and information systems.

FIG. 3 continues the example of FIG. 2A to illustrate the system updating devices with optimized threshold values that may reduce alarms to a manageable number.

FIG. 4 shows a theoretical approach that may be used to simulate alarm frequency using all data captured by all devices over a long time period; this approach may require prohibitive amounts of storage and computation, and the complete data may not be available in some facilities.

FIG. 5 shows an illustrative table schema of alarm summary data that may be captured in one or more embodiments of the invention to enable efficient analysis of alarm thresholds.

FIG. 6 illustrates calculation of the reduction in the number of alarms when an alarm upper threshold is increased using the maximum values stored in alarm summary records.

FIG. 7 illustrates calculation of the reduction in the number of alarms when an alarm lower threshold is decreased using the minimum values stored in alarm summary records.

FIG. 8A shows an illustrative technique for classifying alarms as high (values above an upper threshold) or low (values below a lower threshold) when this data is not available from a device.

FIG. 8B shows an extension of the clustering method of FIG. 8A that also determines the upper and lower thresholds for alarms when this data is not available.

FIG. 9 shows that an estimate of the distribution of alarm maximum values below a current upper threshold may be used in one or more embodiments to calculate an increase in the number of alarms when the upper threshold is decreased (and equivalently an estimate of alarm minimum values may be used to calculate an increase in the number of alarms when a lower threshold is increased).

FIG. 10 shows use of linear regression to estimate the distribution of alarm maximum values.

FIG. 11 shows estimation of the distribution of alarm maximum values based on a sampled or estimated distribution of all values observed by the patient monitoring devices.

### DETAILED DESCRIPTION OF THE INVENTION

A system that efficiently calculates and sets alarm thresholds for patient monitoring devices will now be described. In the following exemplary description, numerous specific details are set forth in order to provide a more thorough understanding of embodiments of the invention. It will be apparent, however, to an artisan of ordinary skill that the present invention may be practiced without incorporating all aspects of the specific details described herein. In other instances, specific features, quantities, or measurements well known to those of ordinary skill in the art have not been described in detail so as not to obscure the invention. Readers should note that although examples of the invention are set forth herein, the claims, and the full scope of any equivalents, are what define the metes and bounds of the invention.

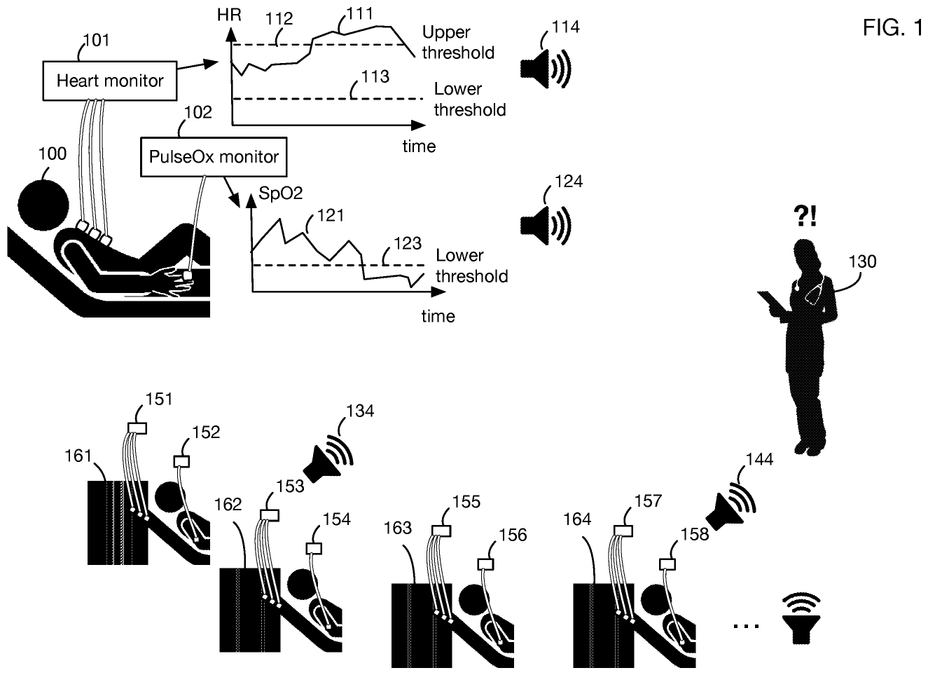

FIG. 1 illustrates a problem that may be addressed by one or more embodiments of the invention. In this example, a medical facility has a large number of patients that are monitored by a large number of patient monitoring devices. Some patients may be monitored by multiple monitoring devices. The facility may be for example, without limitation, a hospital, a skilled nursing facility, a nursing home, a patient's home, an emergency room or urgent care clinic, a surgery center, a physician office, or a collection or network of any of these facilities. The facility may be distributed and may include application of patient monitoring to patients who are at home. Illustrative patient 100 is monitored by a heart monitor 101, which measures heart rate for example (potentially along with many other variables), and pulse oxygen monitor 102, which measures oxygen saturation (SpO2). These devices are illustrative; a monitoring device may monitor any patient parameter or parameters associated with any physiological function or system. Similarly patient 161 is monitored by heart monitor 151 and pulse-ox monitor 152, patient 162 is monitored by heart monitor 153 and pulse-ox monitor 154, patient 163 is monitored by heart monitor 155 and pulse-ox monitor 156, and patient 164 is monitored by heart monitor 157 and pulse-ox monitor 158. For simplicity of illustration, this example shows only five patients; some facilities may have hundreds or even thousands of patients. Also, for simplicity this example shows identical monitoring devices monitoring each patient; in some facilities different patients may have different types of monitoring devices.

Patient monitoring devices may measure a time series of values of one or more associated patient parameters. For example, heart monitor 101 measures time series 111 of heart rate values for patient 100, and pulse-ox monitor 102 measures times series 121 of SpO2 values for patient 100. Similarly, a patient monitor or a hemodynamic monitor may measure the time series of values associated with respiration rate, blood pressures, cardiac output, stroke volume and various vascular and pulmonary resistances. Some or all of the patient monitoring devices may be configured to generate alarms when the values monitored by the device go outside a defined range. For example, device 101 is configured with an upper threshold 112 and a lower threshold 113; when heart rate 111 goes above upper threshold 112 or below lower threshold 113, device 101 may generate an alarm. In the example in FIG. 1, device 101 generates alarm 114 because values 111 go above upper threshold 112. Some devices may be configured with either or both of an upper threshold or a lower threshold. For example, pulse-ox monitor 102 has only a lower threshold 123; because values 121 fall below this lower threshold, device 102 generates an alarm 124. Similarly, devices 153 and 158 generate alarms 134 and 144, respectively, because values measured by these devices go above a corresponding upper threshold or below a corresponding lower threshold. In some devices, alarms may be generated based on more complex analyses than simple comparisons to a threshold; for example, an alarm may be generated if the time series stays outside the threshold(s) for a certain number of samples or if the time series matches a certain pattern.

FIG. 1 illustrates a common problem in environments with multiple patients monitored by patient monitoring devices: alarms may be generated frequently and may overwhelm the clinical staff 130. In part this alarm overload situation may occur because thresholds (such as 112, 113, and 123) may be set to default values or to values that are overly responsive to small deviations from expected norms. While such thresholds may be effective for small numbers of patients, they do not scale well to large facilities with many monitoring devices in operation at once, where clinicians may be constantly responding to alarms even for less serious problems. There are no known systems that systematically analyze alarm thresholds and determine modified thresholds that reduce the total number of alarms.

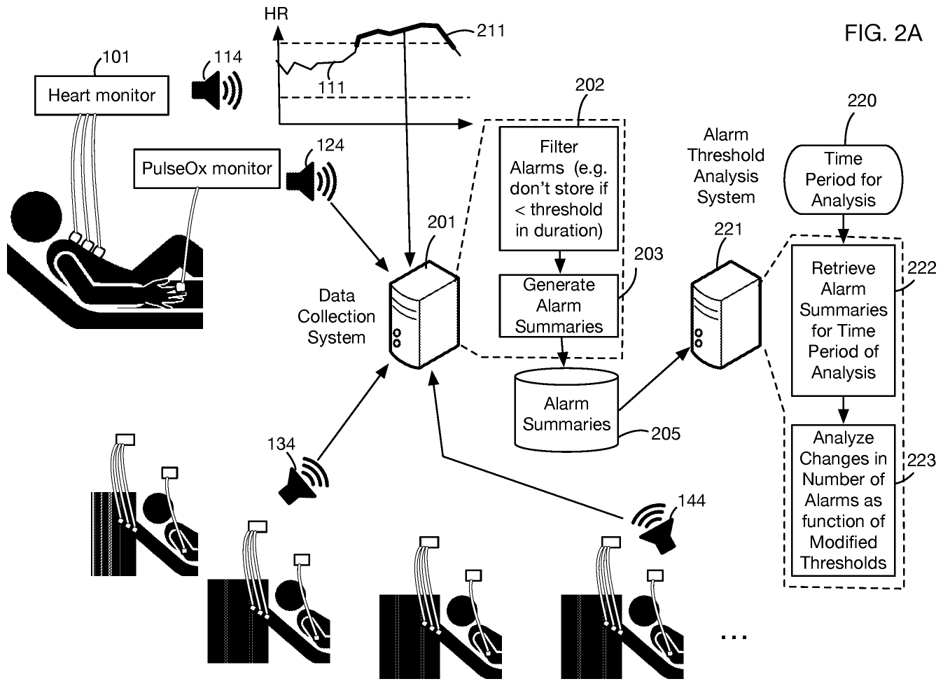

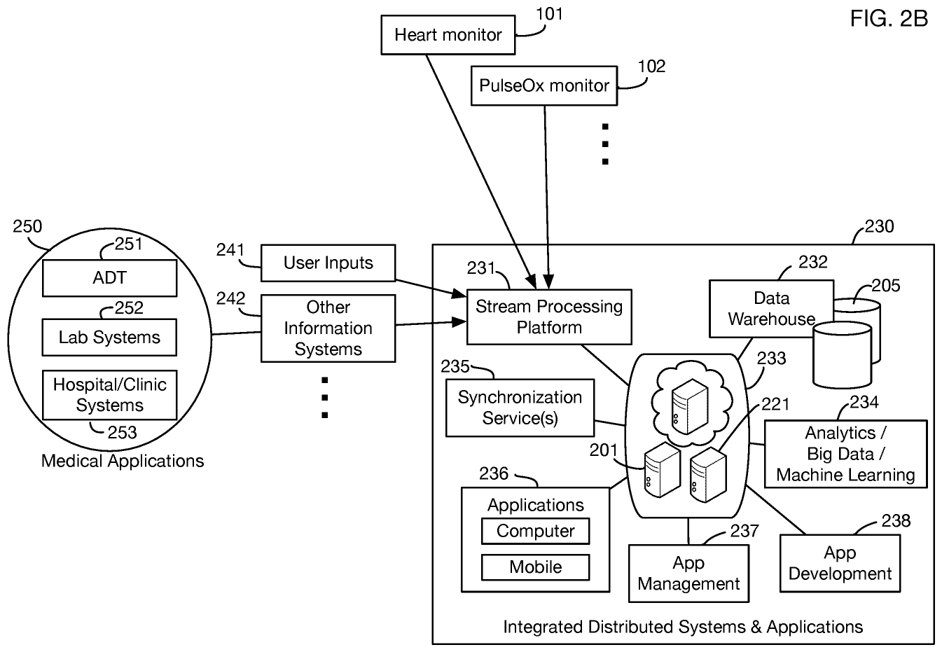

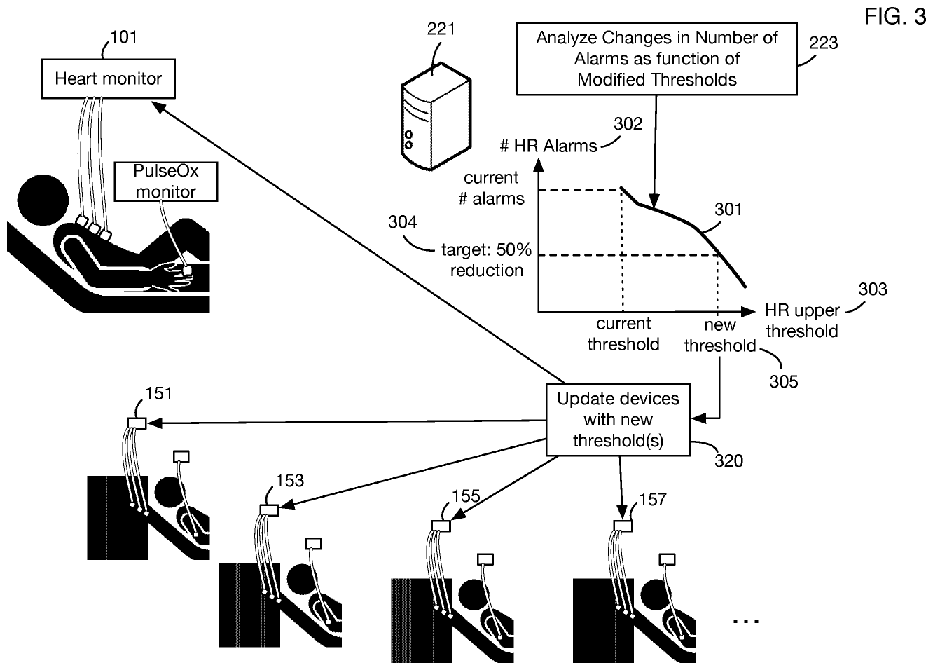

FIGS. 2A, 2B, and 3 show architectural diagrams of an embodiment of the invention that addresses the problem of alarm overload and alarm fatigue illustrated in FIG. 1. FIG. 2A shows an illustrative embodiment of the system that is conceptually divided into a data collection system and an alarm threshold analysis system, although there may be common components shared between these two subsystems. In this embodiment, alarm data is transmitted from patient monitoring devices to a first processor 201 that implements the data collection system. For example, processor 201 may be connected to devices by one or more network links, or data may be collected periodically from devices and uploaded to processor 201. For alarm 114 from device 101, for example, the values 211 above the upper threshold that generated the alarm may be transmitted to processor 201. Processor 201 need not collect all data continuously from all devices; instead, it may receive data when a device generates an alarm, either as the alarm occurs or afterward. Devices may transmit any type of data describing the alarm to the processor; this data generally includes at least the measured values from the device while the alarm is active, as well as the timestamp of the alarm and its duration. Processor 201 collects alarm data from alarms such as 114, 124, 134, and 144. It may perform a filtering operation 202 to remove inconsequential short duration alarm events that represent transient changes or noise in the vital sign. For example, an alarm with a short duration (less than some defined duration threshold value, such as 3 seconds for example) may be due to patient movement or some other transient effect; such alarms may be discarded. In step 203, processor 201 generates an alarm summary for each alarm received (and not discarded by filtering step 202) and stores the summary in alarm summaries database 205. An alarm summary may be a compact representation of the essential data from an alarm that supports subsequent analysis of thresholds, as described below. Processor 201 may collect alarm data and store alarm summaries over a potentially long period of time, such as several weeks, months, or years.

When the facility wants to evaluate possible changes to alarm thresholds, a second processor 221 (which may be identical to first process 201 in some embodiments) that implements the alarm threshold analysis system may obtain a desired time period 220 for the analysis, and then perform step 222 to retrieve the alarm summaries for alarms during that time period from database 205. It may then perform analyses 223 of the alarm summaries to determine expected changes in the number of alarms as a function of modified threshold values. Modified threshold values may then be selected, either manually or automatically, to reduce the number of alarms to a desired level. Analysis 223 also allows the facility to explore the impact of different threshold values and to investigate the tradeoffs between changing thresholds and increasing or reducing the burden of responding to alarms.

Processors 201 and 221 may be any type or types of computers or processors, including for example, without limitation, a server, a laptop, a desktop, a notebook, a tablet, a CPU, a GPU, an ASIC, or a network of any of these devices. Processors 201 and 221 may be the same hardware or they may be different hardware.

FIG. 2B shows an illustrative architecture of hardware and software components that may receive, store, or process device data or other information, and that may incorporate some or all of the processing steps described in FIG. 2A. Data from devices such as heart monitor 101, pulse oxygen monitor 102, or other patient monitoring devices may be streamed or transferred to a transmitted to system 230, which may for example be an integrated, interconnected, and potentially distributed collection of processors, applications, and storage subsystems. Data may be received for example by a stream processing platform 231, or a distributed set of stream processing platforms, which may transform or forward streams to other system components. In one or more embodiments, other data in addition to patient monitoring data may also be streamed or otherwise transferred to system 230, such as data from other information systems 242 and user inputs 241. For example, in a medical application, information systems 242 that may be connected to system 230 may include systems such as ADT (admission, discharge, and transfer) systems 251, laboratory systems 252, and hospital or clinic information systems 253.

The applications and data storage subsystems integrated into system 230 may be executed or managed by one or more processors 233, which may include the system(s) 201 and 221 as well as any other servers or other computers. Any of these systems may be or may have any type or types of processors, including for example, without limitation, desktop computers, laptop computers, notebook computers, CPUs, GPUs, tablet computers, smart phones, servers, customized digital or analog circuits, or networks of any of these processors. Some or all of these systems may be remote from the sites housing devices 101 and 102. Some or all of the systems may be cloud-based resources, such as for example AWS® servers or databases. Data and processing may be distributed among the processors 233 in any desired manner. Illustrative embodiments of system 230 may include any number of stream processing components such as AWS Kinesis® or Apache KAFKA® with KSQL® or SPARK®, database components, computational components, data warehouse, data lake or data hub components, analytics components, and applications components. Applications may be managed by an application management subsystem 237, which may for example manage deployment, distribution of processing across processors, and data interconnections among components. An application development platform 238 may also be connected to the other components of system 230, so that new or modified applications can access streams, data, and component outputs for development and testing.

The stream processing platform 231 (which may be a distributed network of stream processing systems) may provide immediate access to received packets by applications that are part of or connected to system 230. For example, in a medical embodiment these applications may include algorithms for detecting and predicting cardiac arrhythmia, physiological decompensation and diverse types, cardiac and respiratory events, inadequate blood pressure and/or blood oxygen and glycemic instability. System 230 may utilize waveform data to inform clinicians, extract features indicative of patient physiological state (such as heart rate variability), support predictive applications, enable application development, and display results at local and remote locations.

In one or more embodiments, accurate results may necessitate waveform alignment which may be performed by synchronization service(s) 235 as packets are received by the stream processing engine 231.

Data received by stream processing platform 231, or from other sources or subsystems, may be stored in one more databases or other storage systems, which may implement or connect to data warehouses, data lakes, or data hubs 232, such as database 205. System 230 may provide access to data stored in any database, data warehouse, data lake, or data hub, to applications 236, which may include computer-based applications and mobile apps. Stored data or directly streamed data may also be processed by analytical systems 234, which may for example include machine learning and big data analytics. In medical applications, data may be processed in bulk to provide representative data sets for determining models capable of detecting and predicting clinical conditions and events and patient state. Analytics 234 and applications 236 may require synchronization of waveform data; synchronization services 235 may perform this synchronization before storage, upon retrieval from storage, or on streamed data as it is received. A user or subsystem may for example request synchronization of waveforms for a specific patient or for multiple patients over a particular time interval or intervals.

System 230 may also provide application access to data stored in the database, data warehouse, data lake and/or data hub for user consumption for offline viewing, reporting, annotation and chart review. Here, synchronization 235 may be applied to waveforms either prior to insertion into the database or data warehouse or after querying for the desired data subset.

FIG. 3 continues the architectural diagram of FIG. 2A to show an illustrative result of analysis 223. In this example the analysis focuses on heart rate alarms. In one or more embodiments, analysis may address a specific type of alarm from a single type of device, or multiple types of alarms from multiple types of devices. The analysis may for example indicate which types of devices generate the largest number of alarms, and the device types for which changes in threshold values may have the greatest impact on the reducing the total number of alarms. Analysis 223 processes alarm summary data for a desired time period to generate a curve 301 that shows the number of heart rate alarms 302 as a function of potential modifications to the heart rate upper threshold 303. A similar analysis may be done to generate a curve of the number of alarms as a function of a modified lower threshold. This analysis may be used for example to determine a modified threshold value 305 that results in a target reduction 304 in the number of alarms.

In one or more embodiments of the invention, a new threshold value 305 selected by the system (or by a user) may be transmitted in step 320 to one or more of the patient monitoring devices to replace their existing threshold values. In the example in FIG. 3, the new threshold value 305 is transmitted to heart rate monitors 101, 151, 153, 155, and 157 as a replacement for their upper thresholds. In one or more embodiments only a subset of the devices may be updated, and the system may for example prioritize devices that generate more alarms, or devices associated with less critical patients. Updates 320 may replace either or both of an upper threshold and a lower threshold. In some environments, the collection and analysis of alarm data, selection of modified thresholds, and update of devices with new thresholds may be fully automated to form a closed-loop system that iteratively adjusts device behavior to achieve a desired rate of alarms.

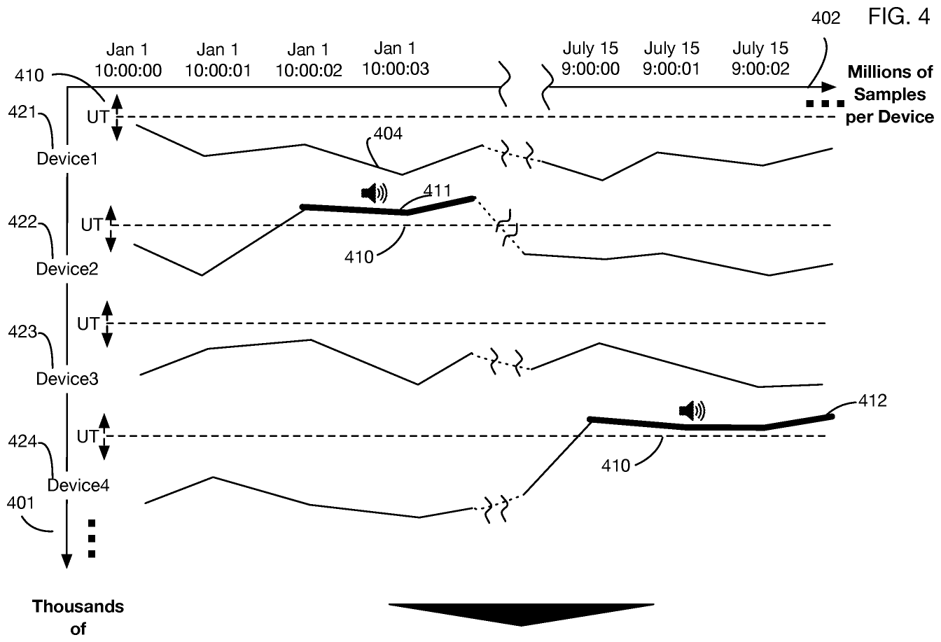

Turning now to the analysis process 223 that determines changes in the number of alarms as a function of modified thresholds, in theory this analysis could perform a comprehensive simulation using all patient data measured by all devices over a period of time. This potential approach is illustrated in FIG. 4, which shows a small portion of a potentially very large table of data captured by a large number of devices 401 over a long time period 402. For example, waveform 404 shows data captured from device 421 at the beginning and at the end of the time period 402. A proposed upper threshold 410 may be compared to the time series of data from every device across the entire time period to identify the alarms that would be generated. (A similar analysis could be performed for lower thresholds.) With this threshold 410, alarms would be generated for example for data 411 of device 422, and data 412 of device 424. This analysis could be repeated over all devices and over the entire time period to count the number of alarms generated. This analysis could be repeated for a large number of proposed upper thresholds to generate the functional relationship between threshold values and the resulting number of alarms.

While the analysis described above with respect to FIG. 4 is theoretically possible, in practice it would require extremely large amounts of storage and computation. With potentially thousands and devices and time periods over months, many billions of data points would need to be stored, and the entire data set would have to be reanalyzed for each possible threshold. Additionally, this comprehensive approach would lead to unacceptable delays in presenting the results to the user. Moreover, in many environments, devices may not store or transmit their data at all times; instead, they may be configured to only store or transmit data captured during an alarm. Consequently, in practice, alarm thresholds are manually modified on each device and the resulting number of alarms is observed over weeks and months in order to identify a beneficial setting. A more efficient approach is needed that supports rapid evaluation of different threshold values without excessive storage or computation. This efficient approach is enabled by embodiments of the invention, which may use alarm summary data to rapidly analyze the effect of changing thresholds on the number of alarms.

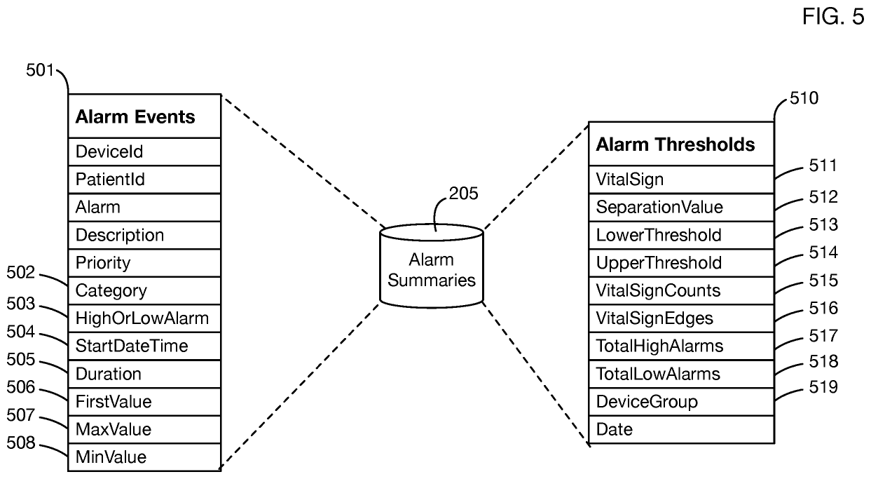

FIG. 5 shows an illustrative schema of an Alarm Events table 501 that may be stored in the alarm summaries database 205. Any data describing any aspect of an alarm may be stored in this table. A Category field 502 may for example indicate the type of alarm, such as a technical alarm or a vital sign alarm. The alarm field may store the name of the alarm such as heart rate alarm or SpO2 alarm. The HighOrLowAlarm field 503 may indicate whether the alarm is a high value that exceeds an upper threshold or a low value that is below a lower threshold. The StartDateTime field 504 may indicate when the alarm starts and the Duration field 505 may indicate how long the alarm was active.

Instead of storing all of the values read by the device while an alarm is active, in one or more embodiments the table 501 may store only selected values that support analysis of threshold changes, as described below. For example, table 501 includes FirstValue field 506 that stores the value of the first sample from the device after an alarm is generated (or concurrent with the generation of the alarm), Max Value field 507 that stores the largest value from the device during the time period of the alarm, and Min Value field 508 that stores the smallest value from the device during the time period of the alarm. One or more embodiments may store additional summary information describing the time series of data that corresponds to device readings during the alarm period.

When the HighOrLowAlarm field 503 is available in an alarm summary record, alarms can be partitioned into “high” alarms that occur when device values exceed an upper threshold, and “low” alarms when device values are below a lower threshold. The effects of changes to upper and lower thresholds can then be considered separately.

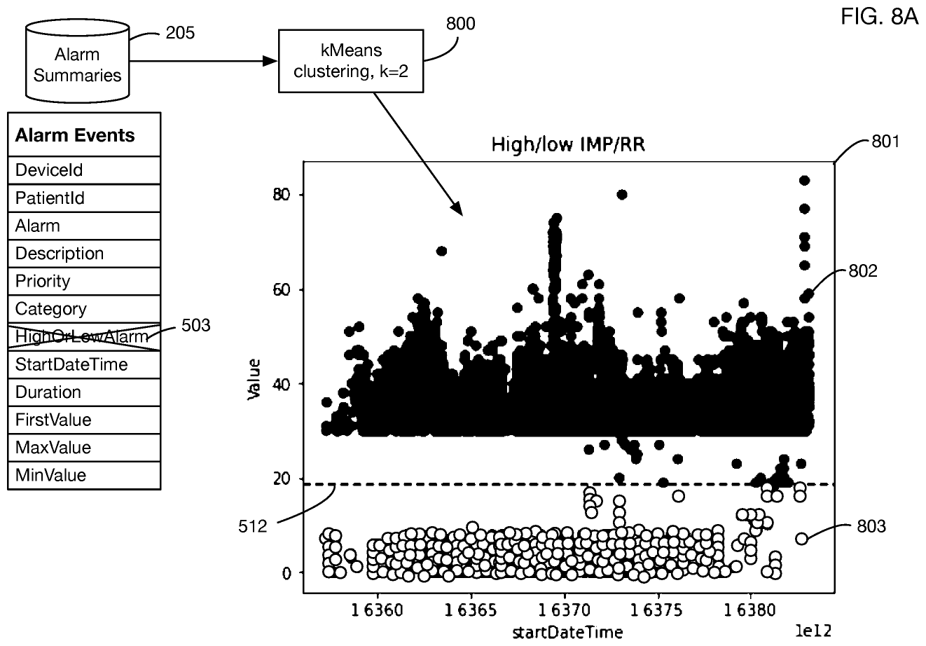

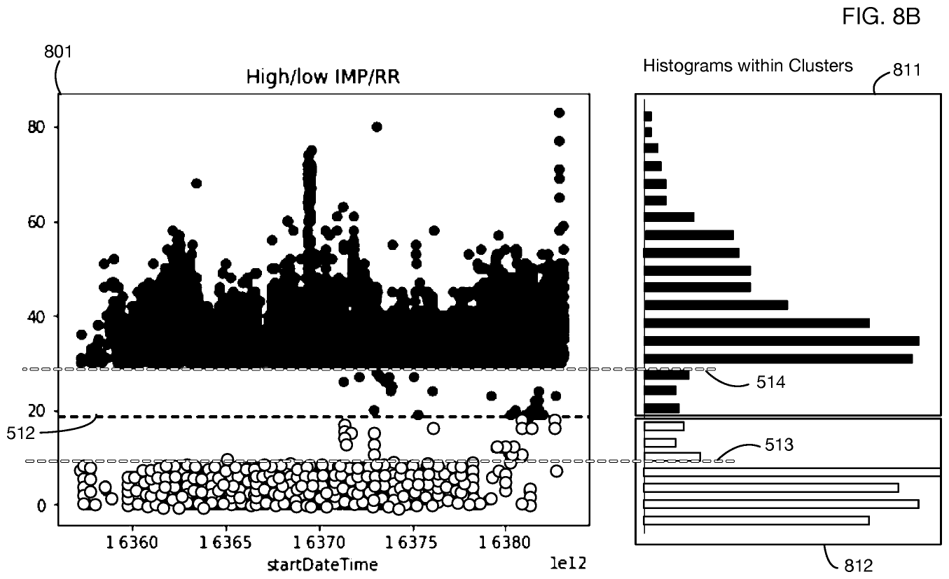

In one or more embodiments the alarms database may also contain an alarm thresholds table 510. This table may contain for example a lower threshold value 513 and an upper threshold value 514 for a group of devices (identified by a device group field 519) that measure a vital sign 511. Fields 519 or 511 may be linked for example to the alarm field or the category field of table 501. As described below with respect to FIGS. 8A and 8B, the threshold values corresponding to a group of devices may need to be estimated or calculated in some situations. The method described in FIG. 8A generates a separation value 512 that may be used to classify alarm events in table 501 as high or low alarms. Fields 515 through 518 may be used for generation of statistical analyses of the number of alarms. For example, alarm statistics may be generated monthly (or for any desired period of time). The alarm events that occurred during this time period may be analyzed as described in FIG. 8B to generate a histogram for each vital sign; the counts and edges of the histogram may be stored in fields 515 and 516, respectively. The total number of alarms classified as high alarms may be stored in field 517, and the total number of alarms classified as low alarms may be stored in field 518.

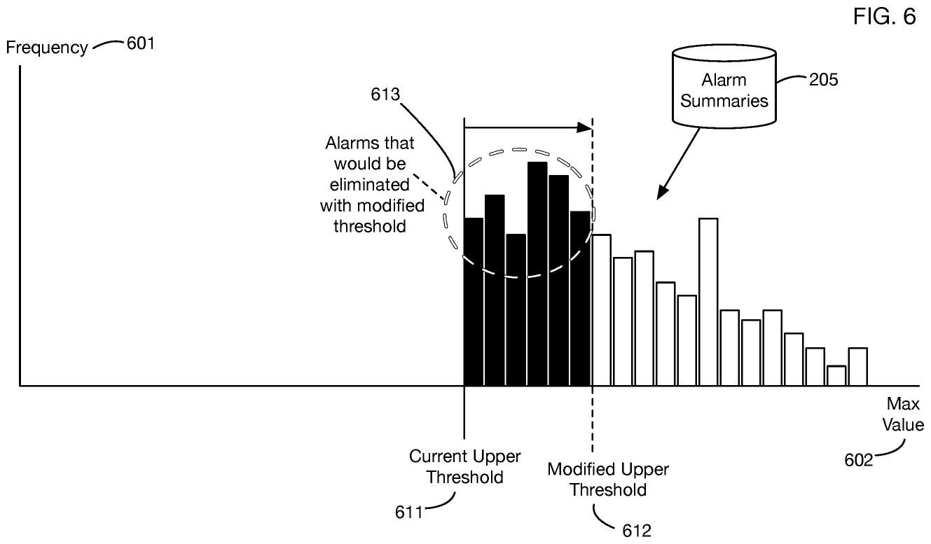

FIG. 6 illustrates how the maximum values in alarm summary records may be used to determine the change in the number of high alarms when an upper threshold is increased. This analysis is based on the principle that all values measured by the device during an alarm are less than or equal to the maximum value; therefore, if the upper threshold is set above this maximum value, no values would have exceeded the upper threshold and the alarm would not have occurred. FIG. 6 shows an illustrative histogram of the frequency 601 of the maximum values 602 in high alarm summary records from a particular type of device (such as a heart rate monitor) for the desired time period obtained from the alarm summaries database 205. (This histogram shows only the “high” alarms for this type of device.) The current upper threshold 611 is below the alarm maximum values, because the data measured during the alarm exceeded the threshold at some point during the alarm. If the upper threshold were increased to a modified value 612, all of the alarms 613 shaded in black would be eliminated. The total frequency of these eliminated alarms is therefore the reduction in the number of alarms when increasing the upper threshold to 612. This analysis can evaluate a range of modified upper threshold values 612 to generate a functional relationship (like curve 301 in FIG. 3) between modified threshold values and the resulting number of alarms.

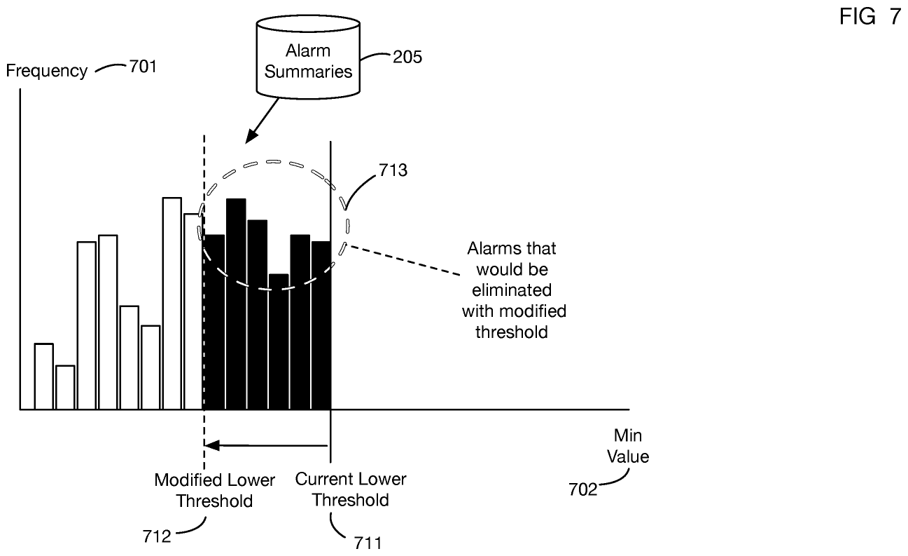

FIG. 7 illustrates a similar analysis for reduction in the number of “low” alarms when a lower threshold is decreased. The histogram of frequency 701 of low alarm minimum values 702 for a specific type of device is generated from alarm summary records over a desired time period obtained from alarm summaries database 205. If the lower threshold is decreased from the current value 711 to a modified value 712, alarms 713 (shaded in black) would be eliminated. This analysis can be performed for a range of modified lower thresholds to generate a functional relationship between modified threshold values and the resulting number of alarms.

In some environments, alarm data transmitted from a device may not include an indication of whether the alarm occurred because values exceeded an upper threshold (a “high” alarm), or because values were below a lower threshold (a “low” alarm). To analyze the effects of modified thresholds, alarms from the device may first be classified as high alarms (exceeding an upper threshold) or low alarms (below a lower threshold). This situation is illustrated in FIG. 8A, where field 503 (HighOrLowAlarm) is missing from at least some of the alarm summaries in database 205. The system may use any type of classifier to assign the value of missing field 503. In one or more embodiments, K-mean clustering may be applied to the vital signs that are observed when an alarm is active. This enables the separation of high and low alarm types. Subsequently, the determined point of separation, together with the histogram of vital signs received during alarms, may be used to calculate the thresholds, as described below with respect to FIG. 8B. FIG. 8A illustrates use of a kMeans clustering algorithm 800, with k=2 (two clusters). Plot 801 shows values of the First Value field of alarm summary records for the relevant device type (plotted against the alarm StartDateTime to spread out the data points for illustration). The points are separated into two clusters, with the upper cluster points shaded black and the lower cluster points shaded white. A separation value 512 between the clusters may be defined for example as the average of the two cluster means. This separation value may be used to classify other alarms as they arrive, with alarms having a first value above the separation value classified as high alarms and alarms having a first value below the separation value classified as low alarms. Use of kMeans clustering is illustrative; one or more embodiments may classify alarms into high or low alarms using any desired algorithm.

In some situations, either or both of the upper and lower thresholds for alarms may also not be available; the system may only obtain information that an alarm occurred, and the values from the device during the alarm may be available. In these scenarios, the first value recorded for an alarm is likely, but not guaranteed, to be near the alarm threshold. The frequency of first values may therefore be used to estimate the upper and/or lower threshold values. This technique is illustrated in FIG. 8B, which continues the example of FIG. 8A. The first values of alarms are clustered into two clusters as described in FIG. 8A, as shown in plot 801. The points shaded black are the high alarms, the points shaded white are the low alarms, and separation line 512 divides the alarms into high and low alarms. Plots 811 and 812 show histograms for the frequency of first values within each cluster, for the high alarms and low alarms, respectively. The obvious discontinuity or rapid change in alarm first value frequencies at value 514 for high alarms and value 513 for low alarms strongly suggests that these values correspond to the upper and lower thresholds, respectively. A specific illustrative algorithm that may be used in one or more embodiments to determine upper and lower alarm thresholds is as follows: First, a second k-means clustering may be performed on each of the high alarms and low alarms separately. The middle of the cluster centroids in each group may be defined as “extreme points”, with the extreme minimum point at the middle of the cluster centroids for the low alarms, and the extreme maximum point at the middle of the cluster centroids for the high alarms. The minimum point for the low alarms may then be set to the point two-thirds of the way from the separation point 512 towards the extreme minimum point [minimum point=separation point-(separation point-extreme minimum point)*⅔]. Similarly, the maximum point for the high alarms may then be set to the point two-thirds of the way from the separation point 512 towards the extreme maximum point [maximum point=separation point+(extreme maximum point-separation point)*⅔]. Using histograms 811 and 812 of alarm first value frequencies (within each group), the mode may then be calculated for the high alarm group as the value with the highest frequency between the separation point and the maximum point, and for the low alarm group as the value with the highest frequency between the separation point and the minimum point. Starting at the separation point, the alarm threshold may be set as the first value higher (for high alarms) or lower (for low alarms) whose histogram count is greater than the frequency of the mode divided by 3. Values below (for high alarms) or above (for low alarms) the threshold will be considered outliers.

The specific algorithm described above to locate upper and lower thresholds is illustrative; one or more embodiments may use any algorithm that for example starts at the separation point and searches upward (for high alarms) or downward (for low alarms) for values that have frequencies indicative of significant numbers of alarms, where values closer to the separation point are outliers with significantly lower frequencies. These threshold frequencies may be based for example on the modes of the high alarm and low alarm frequency distributions, or on any portion of these distributions (as described above for example where the high mode is located between the separation point and the maximum point, and the low mode is located between the minimum point and the separation point). Threshold frequencies to determine the upper and lower thresholds may be calculated as specified fractions of the respective modal frequencies, such as ⅓ of the modal frequency as described above.

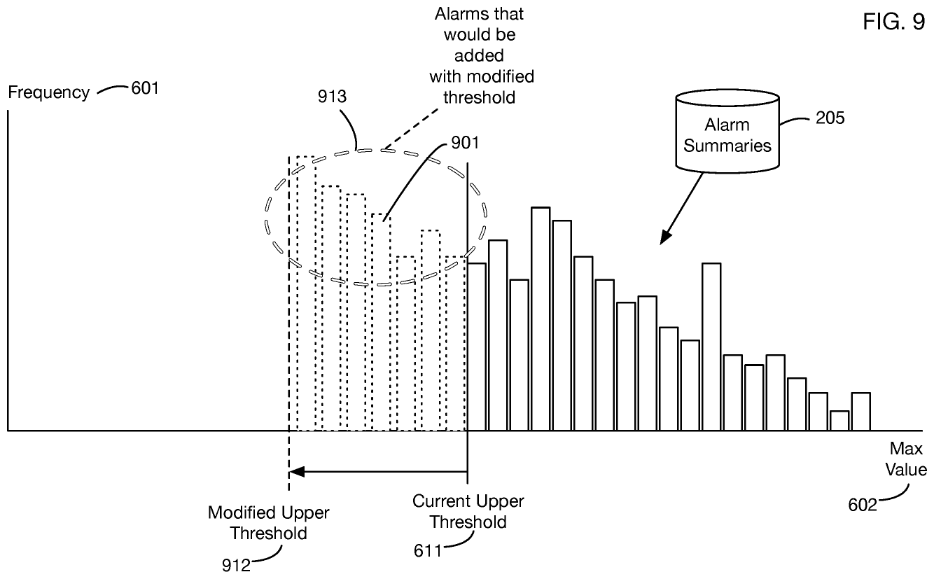

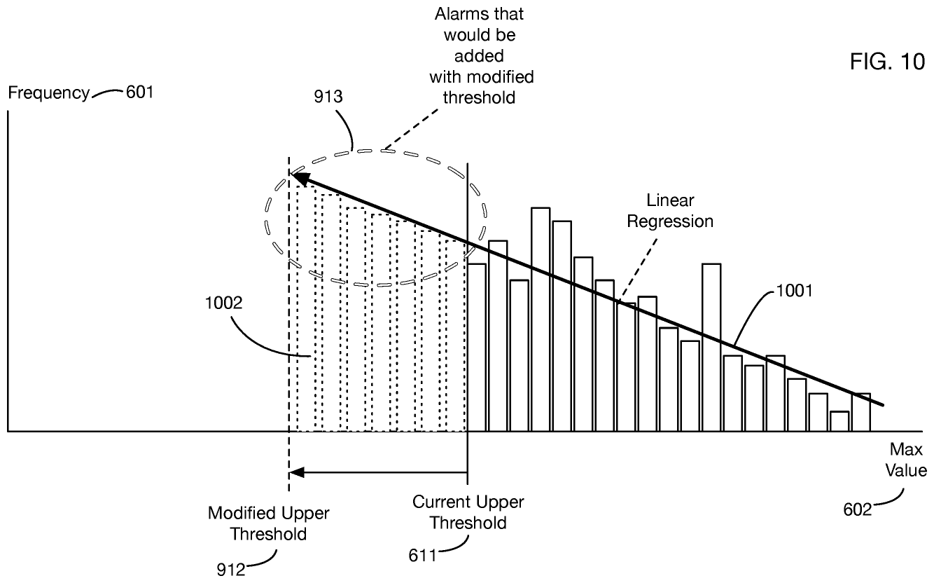

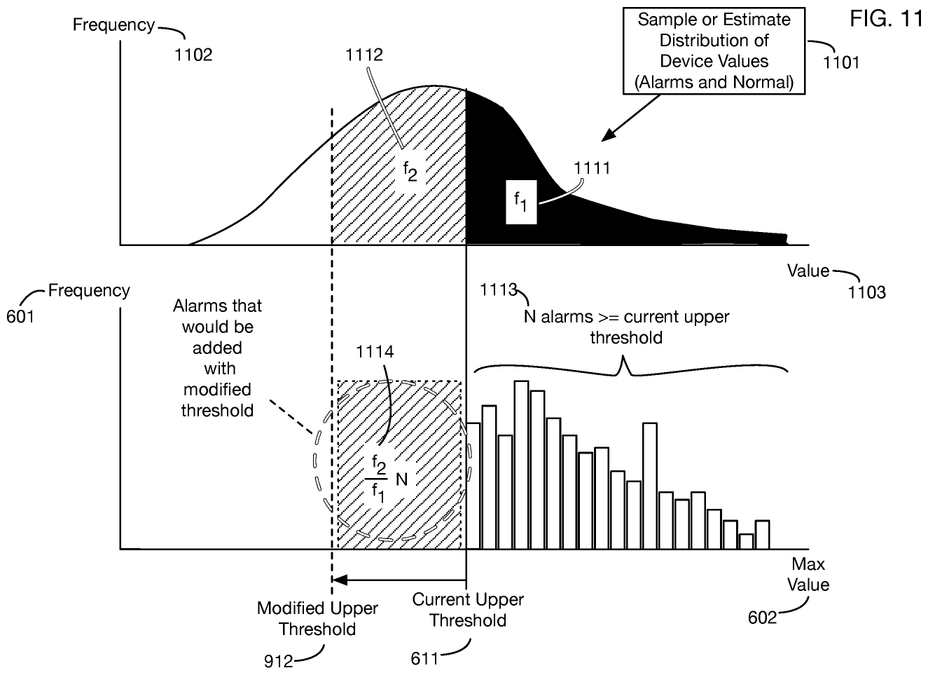

As described above, the maximum values in alarm summaries may be used to determine the reduction in the number of alarms when an upper threshold is increased, and the minimum values in alarm summaries may be used to determine the reduction in the number of alarms when a lower threshold is increased. In some situations, it may be valuable to investigate how sensitive the number of alarms is to decreases in the upper threshold or increases in the lower threshold, both of which may increase the number of alarms. Because this analysis determines how many new alarms would occur, it depends on data that is not captured in the alarm summaries (which record only alarms that did actually occur). FIG. 9 illustrates an approach to estimating the number of additional alarms when an upper threshold is decreased; the analysis for an increased lower threshold is similar. FIG. 9 shows the same histogram of frequency of maximum values from the alarm summaries database (for a specific time period and a specific type of device) as FIG. 6. As upper threshold 611 is reduced to a smaller value 912, some number of additional alarms 913 would occur. The frequencies 901 of these alarms are not available in the alarm summaries database, so they must be estimated. FIGS. 10 and 11 illustrate two different approaches that may be used to estimate these frequencies 901. In FIG. 10, a linear regression line 1001 (or other linear or nonlinear functional estimate) is calculated based on the frequencies of maximum values above the current upper threshold 611 (which are available from the alarm summaries). This line is then extended to the range between the modified threshold 912 and the current upper threshold 611, and the sum of the frequencies in this range is the estimate 913 for the number of alarms added. In FIG. 11, a sample or estimate 1101 is obtained of the frequency distribution 1102 of all of the values 1103 from the device type being analyzed, including both values that have generated alarms and “normal” values that have not generated alarms. An assumption may be made that the ratio of new alarms 1114 to existing alarms 1113 is the same as the ratio of the cumulative frequency 1112 of device values between the modified and current upper threshold to the cumulative frequency 1111 of device values above the current upper threshold. This estimate 1114 of the number of new alarms therefore equals the ratio of frequency 1112 to 1111, multiplied by the number 1113 of current alarms with the current upper threshold.

In one or more embodiments of the invention, calculation of a new alarm threshold may be performed on a patient-by-patient and alarm-by-alarm basis. In the event that one or more individual patients experience an increase in alarms beyond the expected range, the second processor may automatically determine a new threshold to reduce the alarm rate while at the same time maintaining the vigilance intended by the use of alarms. For example, a patient with repeated alarms of low duration may have a baseline vital sign value that is out of the ordinary with respect to the population of normal patients within a particular care area. For example, a patient may have a heart rate that is systematically higher than most patients within a particular unit. Probabilistically, this will lead to an increase in non-actionable heart rate high alarms.

The greater than expected alarms may be determined by comparing the alarm rate associated with a single patient and a specific alarm to that of the past population of patients. For example, if the alarm rate exceeds the average rate plus two times the standard deviation of patient alarm rates, then the observed single patient alarm rate may be classified as elevated.

Another criterion that may considered in automatic alarm threshold determination for a patient is whether the associated vital sign has been stable during the period in which alarms were observed. Stability may be defined in a variety of ways and may be observed for example based upon the trend in the time series of raw vital sign values. To ensure efficiency, stability may be determined for example based upon the disclosed alarm summary database as a consistent pattern of extreme alarm values that differ from the first alarm value by less than a fraction of the population average deviation.

Given the detection of a stable patient, the analysis previously described may be applied to the single patient data to determine a new alarm threshold that yields alarm rate estimates similar to that of the overall patient population. A new alarm threshold may be applied to the bedside monitor of the patient after verification by a clinician who is a system administrator.

While the invention herein disclosed has been described by means of specific embodiments and applications thereof, numerous modifications and variations could be made thereto by those skilled in the art without departing from the scope of the invention set forth in the claims.

What is claimed is:

1. A system that efficiently calculates and sets alarm thresholds for patient monitoring devices, comprising:
a data collection system comprising a first processor coupled to a database and coupled to a multiplicity of patient monitoring devices, wherein
each patient monitoring device of said multiplicity of patient monitoring devices is configured to
obtain a time series of patient data samples;
receive one or more threshold values;
when said patient data samples are outside said one or more threshold values,
generate an alarm; and
transmit alarm data to said first processor, wherein said alarm data comprises said patient data samples while said alarm is active;
said first processor is configured to
generate an alarm summary record associated with said alarm data and store said alarm summary record in said database, wherein said alarm summary record comprises
said alarm;
an alarm start time;
an alarm duration;
a minimum value of said patient data samples of said alarm data; and
a maximum value of said patient data samples of said alarm data; and,
an alarm threshold analysis system comprising a second processor coupled to said database, wherein said second processor is configured to
retrieve alarm summary records over a time period from said database; and,
calculate an expected change in a number of alarms over said time period as a function of one or more modified threshold values based on said alarm summary records.

2. The system that efficiently calculates and sets alarm thresholds for patient monitoring devices of claim 1, wherein said second processor is further configured to
determine a new threshold value of said one or more modified threshold values; and,
transmit said new threshold value to one or more of said multiplicity of patient monitoring devices to replace one or more of said one or more threshold values.

3. The system that efficiently calculates and sets alarm thresholds for patient monitoring devices of claim 2, wherein said second processor is further configured to determine said new threshold value as a modified threshold value that results in a target value of said expected change in said number of alarms over said time period.

4. The system that efficiently calculates and sets alarm thresholds for patient monitoring devices of claim 1, wherein
said second processor is further configured to obtain or calculate a classification of each alarm summary record of said alarm summary records as
a high alarm corresponding to said patient data samples above an upper threshold, or
a low alarm corresponding to said patient data samples below a lower threshold.

5. The system that efficiently calculates and sets alarm thresholds for patient monitoring devices of claim 4, wherein
said calculate said expected change in said number of alarms over said time period comprises
when one of said one or more modified threshold values corresponds to a modified upper threshold, eliminate said alarm summary records having said maximum value of said patient data samples below said modified upper threshold; and,
when one of said one or more modified threshold values corresponds to a modified lower threshold, eliminate said alarm summary records having said minimum value of said patient data samples above said modified lower threshold.

6. The system that efficiently calculates and sets alarm thresholds for patient monitoring devices of claim 4, wherein
said alarm summary record further comprises
a first value of said patient data samples of said alarm data; and,
said calculate the classification of said each alarm summary record comprises
apply a k-means clustering algorithm with two clusters to a dataset comprising said first value of said patient data samples of said alarm summary records;
calculate a separation value as a mean of centroids of said two clusters;
classify said each alarm summary record as said high alarm when said first value of said each alarm summary record is above said separation value; and,
classify said each alarm summary record as said low alarm when said first value of said each alarm summary record is below said separation value.

7. The system that efficiently calculates and sets alarm thresholds for patient monitoring devices of claim 6, wherein said second processor is further configured to calculate one or both of said upper threshold and said lower threshold.

8. The system that efficiently calculates and sets alarm thresholds for patient monitoring devices of claim 7, wherein said calculate one or both of said upper threshold and said lower threshold comprises
calculate a first frequency distribution of first values of said alarm summary records that are classified as high alarms;
calculate a first modal frequency as a frequency of a mode of all or a portion of said first frequency distribution;
calculate a second frequency distribution of first values of said alarm summary records that are classified as low alarms;
calculate a second modal frequency as a frequency of a mode of all or a portion of said second frequency distribution;
calculate said upper threshold as a smallest value above said separation value having a frequency in said first frequency distribution greater than a first fraction times said first modal frequency; and,
calculate said lower threshold as a largest value below said separation value having a frequency in said second frequency distribution greater than a second fraction times said second modal frequency.

9. The system that efficiently calculates and sets alarm thresholds for patient monitoring devices of claim 4, wherein said second processor is further configured to calculate an expected increase in said number of alarms over said time period when said upper threshold is decreased to a smaller upper threshold or said lower threshold is increased to a larger lower threshold.

10. The system that efficiently calculates and sets alarm thresholds for patient monitoring devices of claim 9, wherein said calculate said expected increase in said number of alarms over said time period comprises
obtain or estimate a distribution of said patient data samples;
when said upper threshold is decreased to said smaller upper threshold, calculate said expected increase in said number of alarms as a frequency of said distribution between said smaller upper threshold and said upper threshold divided by a frequency of said distribution above said upper threshold multiplied by said number of alarms; and,
when said lower threshold is increased to said larger lower threshold, calculate said expected increase in said number of alarms as a frequency of said distribution between said lower threshold and said larger lower threshold divided by a frequency of said distribution below said lower threshold multiplied by said number of alarms.

11. The system that efficiently calculates and sets alarm thresholds for patient monitoring devices of claim 9, wherein said calculate said expected increase in said number of alarms over said time period comprises
calculate one or both of
a first distribution of expected alarm maximum values based on extrapolation of a frequency distribution of said maximum value of said alarm summary records;
a second distribution of expected alarm minimum values based on extrapolation of a frequency distribution of said minimum value of said alarm summary records; and
when said upper threshold is decreased to said smaller upper threshold, calculate said expected increase in said number of alarms as a total frequency of said first distribution between said smaller upper threshold and said upper threshold; and,
when said lower threshold is increased to said larger lower threshold, calculate said expected increase in said number of alarms as a total frequency of said second distribution between said lower threshold and said larger lower threshold.

12. The system that efficiently calculates and sets alarm thresholds for patient monitoring devices of claim 11, wherein said extrapolation of said frequency distribution of said maximum value and said extrapolation of said frequency distribution of said minimum value comprises linear regression.

13. The system that efficiently calculates and sets alarm thresholds for patient monitoring devices of claim 1, wherein said first processor is further configured to not store said alarm summary record when said alarm duration is below a duration threshold value.
